(19) United States
(12) Patent Application Publication
(10) Pub. No.: US 2021/0174968 A1
(43) Pub. Date: Jun. 10, 2021

(54) Visualization of Social Determinants of Health

(71) Applicant: 3M INNOVATIVE PROPERTIES COMPANY, St. Paul, MN (US)

(72) Inventors: Ryan C. Butterfield, St. Augustine, FL (US); Paul A. LaBrec, Chippewa Falls, WI (US); Melissa E. Gottschalk, Rancho Santa Fe, CA (US); Christopher L. Hensel, Castleton-On-Hudson, NY (US)

(21) Appl. No.: 17/250,644

(22) PCT Filed: Aug. 26, 2019
(86) PCT No.: PCT/IB2019/057148

§ 371 Date: Feb. 16, 2021

Related U.S. Application Data
(60) Provisional application No. 62/726,834, filed on Sep. 4, 2018.

(51) Int. Cl. G16H 50/30; G06K 9/62

(52) U.S. Cl. CPC G16H 50/30; G06K 9/6247

(57) ABSTRACT

The present disclosure provides systems, devices, methods, and computer-readable media for determining a social determinants of health (SDoH) score. A method can include receiving first data of three or more data types, each data type corresponding to an SDoH domain including economic stability, education, social and community context, health and health care, and neighborhood and built environment, the first data related to a specified geographic region, performing a principal component analysis (PCA) on the received first data to determine respective contribution values for each domain, the contribution values indicating a relative amount of variation the domain contributes to the SDoH score, receiving second data of the three or more data types, the second data related to first sub-geographical region within the specified geographic region, and determining the SDoH score for the first sub-geographical region based on the received second data and the corresponding contribution values.

### BACKGROUND

Health of a population is determined by many factors. Quantification of health is often performed based on medical data, such as number of admissions to a hospital, number of cases of a disease or virus, or the like. Some have even included education factors and economic stability into their quantification of a population health. To date, these quantification techniques are not very granular, they are on prohibitively large geographic regions and are not very robust in their determination of the social impacts on health.

### SUMMARY OF THE DISCLOSURE

The present disclosure provides a computer-implemented method for determining a social determinant of health (SDoH) score, the method including operations. The operations can include receiving first data of three or more data types, each data type corresponding to an SDoH domain including economic stability, education, social and community context, health and health care, and neighborhood and built environment, the first data related to a specified geographic region. The operations can include performing a principal component analysis (PCA) on the received first data to determine respective contribution values for each domain, the contribution values indicating a relative amount of variation the domain contributes to the SDoH score. The operations can include receiving second data of the three or more data types, the second data related to a first sub-geographical region within the specified geographic region. The operations can include determining the SDoH score for the first sub-geographical region based on the received second data and the corresponding contribution values.

The operations can further include standardizing the received first data to a common scale before performing the PCA and wherein the PCA is performed on the standardized first data. The operations can further include, wherein standardizing the received first data includes performing a z-transformation on the received first data. The operations can further include standardizing the determined SDoH score to a specified scale.

The operations can further include, wherein the specified geographical region is comprised of a plurality of disjoint sub-geographical regions including the first sub-geographical region, receiving the second data includes receiving data for each sub-geographical region of the plurality of sub-geographical regions, and determining the SDoH score includes determining respective SDoH scores for each of the sub-geographical regions. The operations can further include, encoding the determined SDoH scores by color and causing a display to provide a view of the specified geographical region with each of the sub-geographical regions colored consistent with the encoding.

The operations can further include, wherein the data type corresponding to the health and healthcare domain includes a value indicating a proportion of a population in the sub-geographical region that has health insurance. The operations can further include, wherein the data type corresponding to the neighborhood and built environment includes data indicating one or more of how accessible healthy food is within the sub-geographical region, a quality of housing available within the sub-geographical region, air quality within the sub-geographical region, water quality within the sub-geographical region, or a relative amount of distressed or underserved geographies within the sub-geographical region. The operations can further include, wherein the data type corresponding to the social and community context includes an indication of the number of people living in the sub-geographical region. The operations can further include, identifying the SDoH score or corresponding data corresponding to an individual user and identifying a diagnosis, treatment, or risk of re-admission based, at least in part, the SDoH score or corresponding SDoH data.

The present disclosure further provides a device or system configured to perform the operations. The present disclosure further provides at least one machine-readable medium including instructions that, when executed by a machine, configure to the machine to perform the operations.

There are various advantages to various embodiments of the present disclosure. For example, according to various embodiments, the SDoH score can be more granular than other attempts at generating an SDoH score. Since the SDoH is more granular, its relevance to individual or smaller groups people is more well-known. An SDoH score, in accord with embodiments, is relevant to anyone who lives within an atomic geographic region corresponding to a minimum granularity of an SDoH score. The SDoH score can be at about a census tract or neighborhood granularity. Using a technique like PCA more accurately models the real value of the SDOH data at a more granular level than previously available, such as at a census tract level.

The SDoH score provides several improvements over prior SES scores, which used only SES measures such as income and education. Overall, the proposed PCA SDoH score shows greater granularity, more precise accountability of variation, more accurate scoring, and a broader range of measures than its predecessors.

### BRIEF DESCRIPTION OF THE FIGURES

The drawings illustrate generally, by way of example, but not by way of limitation, various embodiments discussed in the present document.

FIG. 1 illustrates, by way of example, a diagram of an embodiment of a method for determining an SDoH score using PCA.

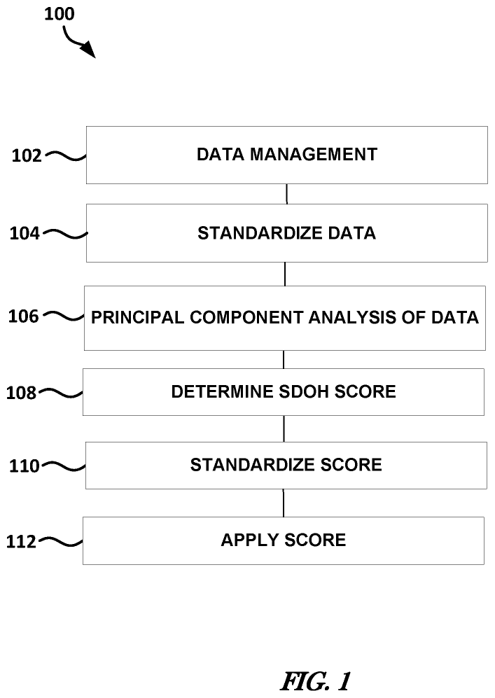

FIG. 2 illustrates, by way of example, a diagram of an embodiment of a system for determining an SDoH score using PCA.

FIG. 3 illustrates, by way of example, a diagram of an embodiment of an SDoH map.

FIG. 4 illustrates, by way of example, a diagram of an embodiment of a method that includes SDoH data (e.g., an SDoH score) in an individual's clinical risk assessment.

FIG. 5 illustrates, by way of example, a block diagram of an example of a device 400 upon which any of one or more processes (e.g., methods) discussed herein can be performed.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for description only and not of limitation. Any use of section headings is intended to aid reading of the document and is not to be interpreted as limiting; information that is relevant to a section heading may occur within or outside of that section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

According to various embodiments of the present disclosure a score indicative of Social Determinants of Health (SDoH) of a specified geographical region can be determined using a Principal Component Analysis (PCA) on social data. As used herein “social data” means data regarding parameters that affect persons socially and affect their health either directly or indirectly. Herein, health means the overall health of a person, including financial health, mental health, physical health, or the like.

The Centers for Disease Control and Prevention (CDC) has implemented an initiative called Healthy People 2020. This initiative defines SDoH domains, domains that are social, but are linked to overall health of a person. These domains include the typical, well-known domains of economic stability and education, as well as lesser-known SDoH domains of social and community context, health and healthcare, and neighborhood and built environment.

In various embodiments, an SDoH score of a geographic region is determined using data from some or all the SDoH domains. This score is more accurate than prior SDoH scores in that it is based on a more universal view of SDoH. The score can be more granular than other attempts at generating an SDoH score. Since the SDoH is more granular, its relevance to people is more well-known. An SDoH score, in accord with embodiments, is relevant to anyone who lives within an atomic geographic region corresponding to a minimum granularity of an SDoH score. The SDoH score can be at about a census tract or neighborhood granularity. The census tract is an area roughly equivalent to a neighborhood established by the Bureau of Census. A census tract generally encompasses a population between about 2,500 to about 8,000 people.

FIG I illustrates, by way of example, a diagram of an embodiment of a method 100 for determining an SDoH score using PCA. The method 100 as illustrated includes data management, at operation 102; data standardization, at operation 104; PCA of the standardized data, at operation 106; determination of an SDoH score, at operation 108; and applying the SDoH score, at operation 110.

The operation 102 includes accessing databases storing data of data types corresponding to SDoH domains, The data can be stored in one or more databases, such as can be publicly accessible over the Internet, accessible with a username and password, or otherwise available for use. Examples of entities that provide public access to data include the United States (US) Census Bureau, US Department of Agriculture (USDA), US Geological Survey (USGS), Centers for Disease Control and Prevention (CDC), or the like. For example, the American Community Survey (ACS), a branch of the US Census Bureau provides public access to results of the surveys they conduct. Data regarding poverty, income, employment/unemployment, public assistance, access to capital, high school graduation, enrollment in higher education, language and literacy, early childhood education, insured/uninsured, and quality of housing, among others, is available through the ACS. This information is publicly available through the ACS website (hops://www.census.gov/acs/www/data/data-tables-and-tools/, last accessed Aug. 29, 2018). In another example, the Economic Research Services branch of the USDA produces the Food Access Research Atlas (FARA) and makes that data available to the public through a website (https://www.ers.usda.gov/data-products/food-access-research-atlas/, last accessed Aug. 29, 2018). Data regarding access to healthy foods and population density is available through the FARA. In another example, the US Environmental Protection Agency (EPA) performs the National Air Toxics Assessment (NATA) and makes that data available through a website (https://www.epa.gov/national-air-toxics-assessment, last accessed Aug. 29, 2018). Data regarding air quality is available from NATA. In yet another example, data collected to help ensure conformity to the Community Reinvestment Act (CRA) that is overseen by the Federal Financial Institutions Examination Council (FFIEC), is provided through the FFIEC website (https://www.ffiec.gov/cra/distressed.htm, last accessed Aug. 29, 2018). Data regarding underserved geographies is provided through CRA.

The SDoH domains include, for example, economic stability, education, social and community context, health and healthcare, and neighborhood and built environment. The data can be a measure of one or more of the domains. For example, economic stability can be measured by poverty level data (e.g., data indicating a percentage or number of people in a geographical region above or below a poverty line), income amount data, employment level data (e.g., data indicating a percentage or number of people in a geographical region with a full-time job), unemployment data (e.g., data indicating a percentage or number of people in a geographical region without a full-time job), public assistance data (e.g., data indicating a percentage or number of people in a geographical region that receive some form of financial assistance from the public (e.g., state, county, city, national government entity)), access to capital data (e.g., data indicating a percentage or number of people in a geographical region that have access to money from a bank, family, friends, or other source of monetary funds). Education can be measured by, for example, high school graduation rate (e.g., data indicating a percentage or number of people in a geographical region that have graduated high school in a specified period of time), enrollment in higher education (e.g., data indicating a percentage or number of people in the geographical region that have enrolled in post-secondary education in a specified period of time), language or literacy rate (e.g., data indicating a number of languages spoken (on average) per person in the geographical region, a number or percentage of people that can read or write at a specified grade level, or the like), and early childhood education (e.g., data indicating a percentage or number of people in the geographical region that are enrolled in pre-kindergarten schooling). Social and community context can be measured by, for example, population density data (e.g., data indicating the number of people in a geographical region is above or below a threshold amount relative to a size of the geographical region). Health and healthcare can be measured by insured data (e.g., data indicating a percentage or number of people in a geographical region that have health insurance), uninsured data (e.g., data indicating a percentage or number of people in a geographical region that do not have health insurance), access to a hospital or other healthcare facility data (e.g., data indicating a percentage or number of people in a geographical region that live within a specified distance of an urgent care, walk-in clinic, hospital, or other facility at which they can receive healthcare), or diagnosis related group (DRG) information. Neighborhood and built environment can be measured by, for example, data indicating access to healthy foods (e.g., data indicating how much of the geographical region is in a food desert), quality of housing data (e.g., data indicating a number of physical deficiencies in housing in the geographical region (on average, overall, or the like)), environmental conditions data (e.g., water or air quality data indicating an amount of toxins in the water or air), or underserved geographies data (e.g., population loss, poverty increase, and unemployment increase (employment decrease) are all indicators of an underserved geography).

The operation 102 can further include one or more of variable reduction, collapsing of variables, formation of indicators, or formatting the data into a data set suitable for PCA. Variable reduction, sometimes called dimensionality reduction, includes reducing the number of random variables for consideration. The variable reduction typically includes feature selection and feature extraction to obtain a set of principal variables. Typically, variables with a larger proportion of missing values can be dropped to reduce a burden on further processing. Collapsing of variables includes altering data to a common scale. For example, weekly data can be collapsed to monthly data, such as by combining multiple weekly data. In another example, data that is more granular geographically can be collapsed to data on a larger geographical region. Formation of indicators includes collapsing of categorical levels within a variable or creation of a new variable with two or more categories aligned to thresholds of a continuous variable. Formatting the data into a data set suitable for PCA includes selecting and merging variables from different data sets into a dataset formatted for analysis.

The operation 104 can include adjusting the data retrieved at operation 102 to a specified range of values, a specified format, organizing the data in a specified manner (e.g., by domain, data type, or the like), or the like. PCA is sensitive to the scaling of the data input thereto. Standardizing the data can help reduce the possibility that a variation in a variable unduly influences a corresponding weight associated with the variable by the PCA process. In one or more embodiments, a z-score transformation (sometimes called standardization or auto-scaling) can be used to standardize the data. The z-transformation alters the data to be mean zero (0) and standard deviation of one (1).

The operation 104 can further include creating analysis data sets. The analysis data sets can include the standardized data pruned to include only variables for analysis. The variables for analysis can be split up by geographical region (e.g., census tract, neighborhood, county, city, state, or the like). The standardized data can then be stored as a data set for each geographic region of interest.

The operation 106 includes performing a PCA on the standardized data, given the analysis data set of the standardized data. PCA is a multivariate statistical technique. PCA identifies correlated/uncorrelated clusters of variables. The clusters are formed based on correlations structures and allow for estimates of variance explained in the variables. PCA is often classified with factor analysis and latent variable analysis. PCA is employed to determine the relationships which exist within large data sets by forming a series of linear combinations of the variables. These combinations are then put into vector form, which leads to a reduction in the dimensions of the data set. Only non-orthogonal vectors are summed to produce a score, as orthogonal vectors are geometrically restricted from being summed. PCA retains only the most significant components, such as by forming clusters of variables, and uses this information and the correlations from the components to create a weight, which is then used in conjunction with the data to create a score. This technique falls under the label of unsupervised machine learning and can be used to identify clusters of data/variables in a multidimensional space.

Reducing the dimensions of a data set can be the primary goal of PCA, though that is not the primary goal of PCA for embodiments. The primary objective of PCA for embodiments is to extract linear regression weights from principal components of the data, which identify the primary sources of variability in the data.

As previously discussed, PCA is a statistical procedure. PCA uses an non-orthogonal transformation to convert a set of observations of possibly correlated variables into a set of linearly uncorrelated variables called principal components. If there are n observations with p variables, then the number of distinct principal components is the lesser of p and n−1. The first principal component has the largest possible variance (accounts for as much of the variability in the data as possible), and each succeeding component has the highest variance possible under the constraint that it is non-tangential to the preceding component(s). The number of components included in the scoring technique can be determined by the model, with the condition that variance is greater than one (1). The coefficients, sometimes called weights, from the PCA can be stored (e.g., in a file) for later access.

Operation 108 includes determining the SDoH score based on regression coefficients (sometimes call “weights”) of the principal components. The operation 108 can include determining a weighted sum of the variables (a specific piece of data). The scores can be associated with standardized data, so that the score can be calculated once and used as many times as desired. In one or more embodiments, the scores can be standardized (e.g., operation 110 can be performed on the scores) before associating them with the standardized data.

The coefficients of the PCA, in embodiments, are used to develop a scoring algorithm which accounts for the correlational structures underlying the SDOH domains. The regression coefficients taken from the principal component(s) allow a system of weights to be incorporated into the SDOH model. A non-orthogonal rotation strategy can be used to help overcome geometric restrictions on the mathematical operations for vectors. For example, vectors at right angles cannot be summed according to tenants of Euclidean geometry. Since the data are representative of current socioeconomic and demographic conditions in a given community, calculating SDOH scores over a geographic area can represent a more accurate system of measurement of SDoH for a given community (e.g., a geographical area, such as a census tract, neighborhood, city, county, state, or the like).

The operation 110 can include converting SDoH scores to a common scale, such as a scale that is more readily understandable to a human. The scale can include, for example, a number in the range 0 to 100, 0 to 1, or the like. In the scale 0 to 100, a score of 0 indicates that all SDoH data indicates there is no redeeming health value to the social infrastructure of the geographical region and a score of 100 indicates that there is no improvement to be made to the social infrastructure of the geographical region to improve their health.

The operation 110 can include adjusting the standardized to a positive scale. The orientation of the vectors representing the components from the PCA can be in directions which do not contextually align with a desired interpretation. By adjusting all the scores to be positive, the interpretation can be more contextually aligned. After the adjustment, previously positive scores can be greater than previously negative scores, but all scores can be greater than (or equal to) zero. After adjusting the score to be strictly positive, the scores can be scaled to a desired range. An example of a desired range includes [0, 100]. These scores can be associated with corresponding data.

The operation 112 can include a variety of operations. For example, the SDoH score can inform a community (a group of people in a specified geographic region) that their social infrastructure can be detrimental to their own health. The SDoH score can be broken down to further inform the community which domains of SDoH are harming the community the most. The community can then use that information to develop a plan to curb the effects of social determinants on their health.

In another example, two patients with similar clinical risk profiles who live in separate geographical regions with different SDoH scores may have different risks of hospital readmission after discharge. The SDoH score can indicate the amount of social support available for recovery of the patient. An SDoH score at a census tract level can be used along with an SDoH score at an individual level to create categories of ‘social risk’ that could be applied post hoc to clinical risk. Such a combination can further stratify patients for risk of adverse outcomes, such as hospital readmission. The inclusion of SDoH in individual clinical risk assessments is discussed in further detail elsewhere herein.

In yet another example, the SDoH score can be determined for each of a plurality of disjoint sub-regions (e.g., counties, census tracts, neighborhoods, states, country, or the like) of a geographical region (e.g., a city, county, state, country, continent, or the like). The scores for each sub-region can be encoded by color. A view of the larger geographical region can then be displayed with the sub-regions colored consistent with the encoding The scores, in one or more embodiments, can be geographically mapped along census tracts. Such a view provides a user with a quick view to discern which geographical areas need improvement in their social circumstances. The user can discern, by the color, pattern, symbol, or other encoding on the geographical regions the SDoH score. The score can indicate how much the social circumstances and programs of the geographical region are working for or against the health of the persons residing in the geographical region.

The operation 112 can include using the SDoH score as a variable in other health services analyses. Joining the SDoH score as part of another analysis can be done using one or more of geocoding (for individual linked census tracts or other geographical regions) and outcomes of interest. The SDoH score can be normalized so that it has a continuous normal distribution which allows for flexibility for inclusion in other statistical models or other analyses.

The operation 112 can include using the score for influencing hospital discharge planning, managing chronic patients, delivering proactive primary care, reducing emergency room utilization, and others. Having an aggregate measure of a patient's social risk, such as can be provided by the SDoH score, can assist various aspects of care management.

As previously discussed, the SDoH score can be at a neighborhood or other level. The SDoH score can provide useful information on potential person characteristics of local areas residents within the service areas of payers, providers, governments, community-based organizations, or the like.

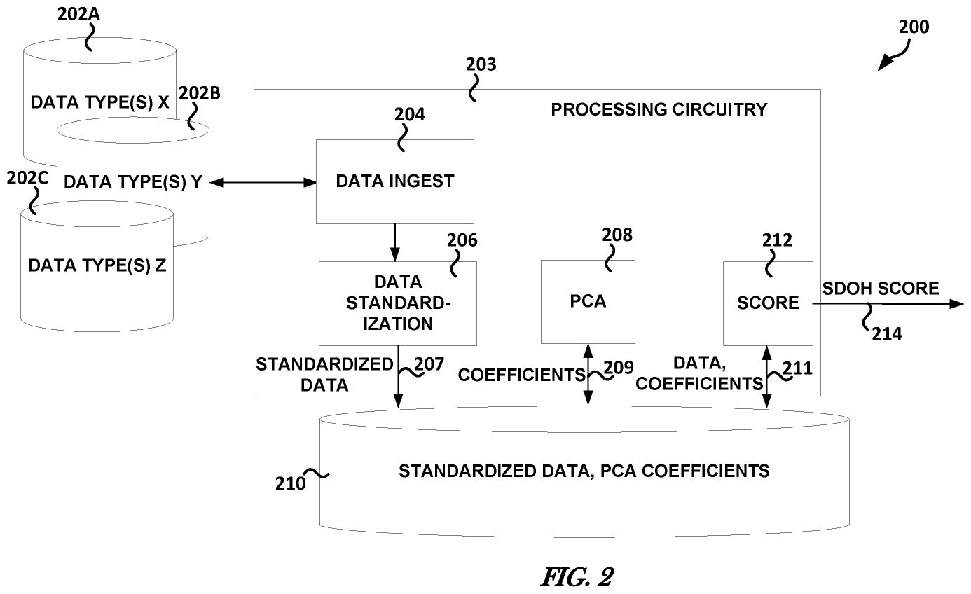

The SDoH scores can be aggregated into an SDH database (see FIG. 2). These SDoH scores can be available for other research models, such as health services research models. The SDoH scores can be used as one or more variables in various health services research analyses, such as can include geospatial healthcare utilization and payment analysis.

FIG. 2 illustrates, by way of example, a diagram of an embodiment of a system 200 for determining an SDoH score using PCA. The system 200 as illustrated includes databases 202A, 202B, and 202C that store data of data types associated with an SDoH domain, processing circuitry 203 to perform the operations of the method 100, and an SDoH database 210 to store data. associated with performing the method 100 and results obtained therefrom.

The databases 202A-202C can be accessible through the Internet or other network. The databases 202A-202C can include data of specified data types stored thereon. Each of the data types can be associated with a specified SDoH domain. The data types can include those previously discussed (e.g., poverty level data, income amount data, employment level data, unemployment data, public assistance data, access to capital data, high school graduation rate, enrollment in higher education, language or literacy rate, early childhood education, population density data, insured data, uninsured data, access to a hospital or other healthcare facility data, access to healthy foods, quality of housing data, environmental conditions data, or underserved geographies data, or other data indicative of an SDoH domain (e.g., economic stability, education, social and community context, health and healthcare, and neighborhood and built environment). Locations to access this data are discussed previously.

The processing circuitry 203 can include one or more electric or electronic components configured to perform the operations of the method 100. The electric or electronic components can include one or more central processing units (CPU), graphics processing units (GPU), field programmable gate arrays (FPGA), application specific integrated circuits (ASIC), transistors, resistors, capacitors, inductors, diodes, rectifiers, regulators, power supplies, memories, logic gates (e.g., AND, OR, XOR, negate, buffer, or the like), multiplexers, switches, oscillators, analog to digital converters, digital to analog converters, or the like. The electric or electronic components can be coupled to one another to form one or more circuits. Different circuits of the processing circuitry 203 can be configured to perform different operations of the method 100. In some embodiments, a single circuit can be configured to perform multiple operations of the method 100, such as to perform two more of the operations 102, 104, 106, 108, 110, and 112. In some embodiments, the circuits can be configured in a networked or distributed architecture, such that multiple circuits perform a portion of an operation of the method 100. In some embodiments, the method 100 can be implemented using a memory (e.g., a machine-readable medium) that includes instructions stored thereon that are executable by a machine (e.g., one or more of the circuits). The instructions, when executed by the machine, configure the machine to perform the operations of the method 100. The instructions, in combination, can form a program code for implementing the method 100.

The processing circuitry 203 can be configured to implement data ingest operations, data standardization operations, PCA, and scoring operations. The circuitry that performs the data ingest operations is called data ingest circuitry 204. The circuitry that performs the data standardization operations is called data standardization circuitry 206. The circuitry that performs the PCA operations is called PCA circuitry 208. The circuitry that performs the scoring operations is called scoring circuitry 212.

The data ingest circuitry 204 can perform the operation 102 of the method 100. The data ingest circuitry 204 can retrieve data from the databases 202A-202C. The data ingest circuitry 204 can perform one or more of variable reduction, variable collapsing, or the like. The ingested data can be provided to the data standardization circuitry 206. In one or more embodiments, the ingested data can be provided to the database 210, such as by the data ingest circuitry 204.

The data standardization circuitry 206 can perform the operation 104 of the method 100. The data standardization circuitry 206 can perform a z-transformation on the ingested data, such as to produce standardized data 207. The standardized data 207 can be provided to the database 210.

The PCA circuitry 208 can perform the operation 106 of the method 100. The PCA circuitry 208 can perform a PCA on standardized data, such as from the database 210 or the data standardization circuitry 206. The PCA circuitry 208 can provide coefficients 209 that are produced as a result of the PCA to the database 210 or the scoring circuitry 212.

The scoring circuitry 212 can perform one or more of the operations 108 and 110. The scoring circuitry 212 can determine an SDoH score 214 based on data and coefficients 211 from the PCA circuitry 208 or the database 210. The SDoH score 214 can be stored on the database 210. The SDoH score 214 can be used in an application, such as an application discussed regarding the operation 112.

The SDoH database 210 can include one or more of ingested data (data before it is standardized by the data standardization circuitry 206), standardized data, PCA coefficients, SDoH score, geolocation data (a value indicating a geographic region to which the data on the database corresponds), date or time associated with the data, or the like. The data on the database can be indexed by geolocation indicator, time, a combination thereof, or the like.

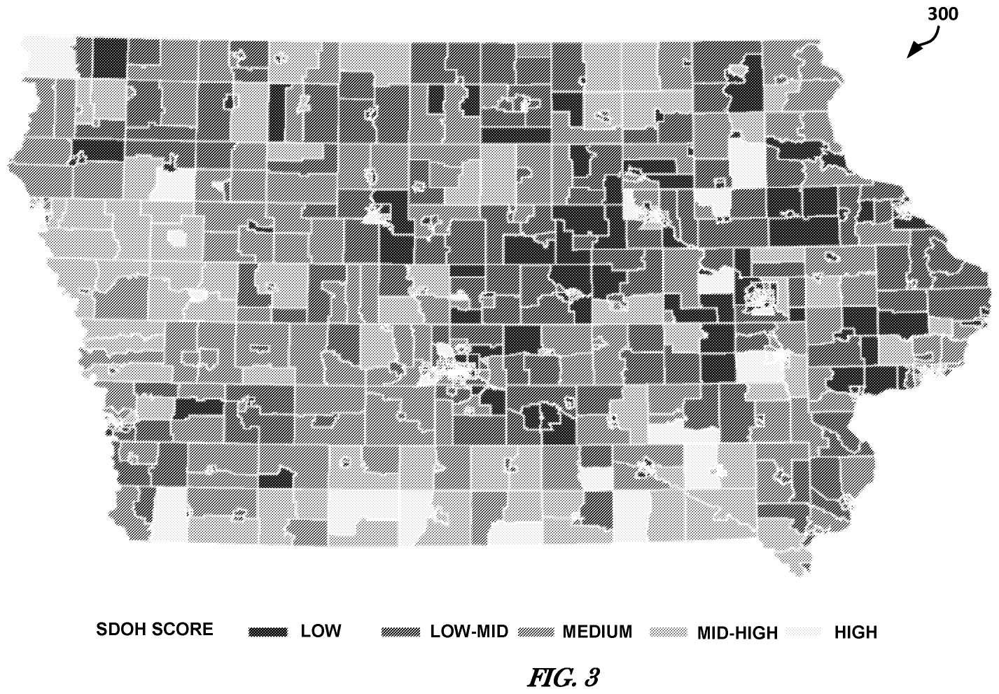

FIG. 3 illustrates, by way of example, a diagram of an embodiment of an SDoH map 300. The SDoH map 300 is of a larger geographic region (a state in the example of FIG. 3) with sub-geographic regions (census tracts in the example of FIG. 3) encoded by color. Each color indicates a different range of SDoH scores. In the example of FIG. 3, a darker color indicates a higher score. To create the SDoH map 300, data of a variety of data types is gathered for each of the census tracts in an example state. For each census tract the data is standardized to a largest geographical level (e.g., a state, country, county, or the like), PCA determines the coefficients, a score is determined based on the data and the coefficients, and the score is encoded to a color. The resulting SDoH map 300 provides a user with a convenient view of the different geographic regions of a state and their relative SDoH scores. The user can then determine, for example, the SDoH score of the geographic regions they inhabit, geographic regions that could use the most help in terms of improving social circumstances of people that inhabit those geographic regions, or other application discussed herein.

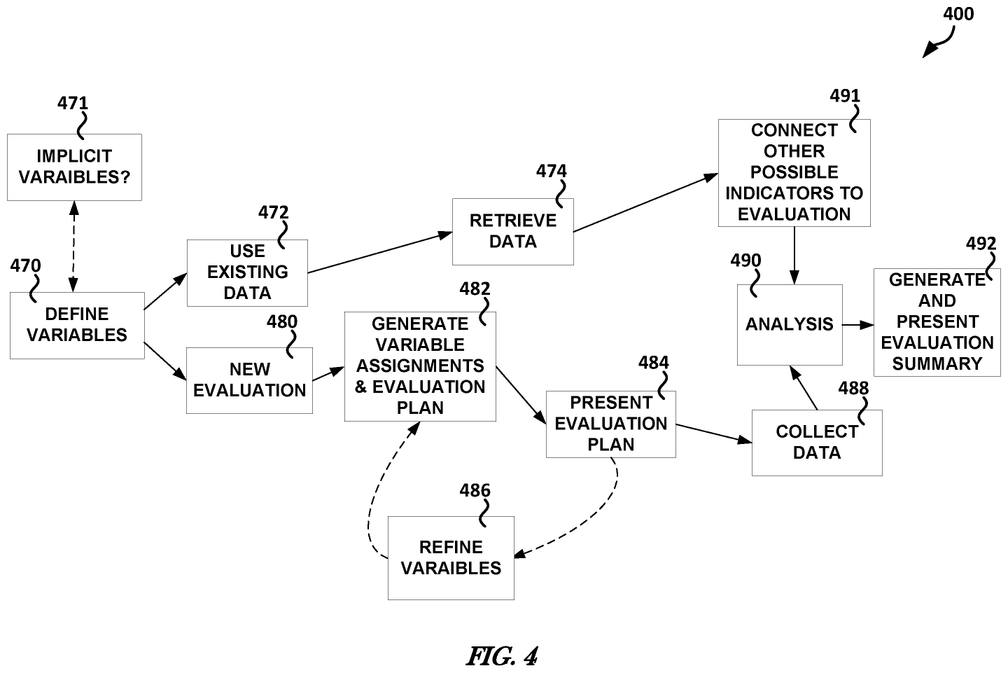

FIG. 4 illustrates, by way of example, a diagram of an embodiment of a method 400 that includes SDoH data in an individual's clinical risk assessment. A user can define independent and dependent variables at operation 470. Independent variables define a medical context, such as a patient who is newly diagnosed as diabetic, whereas dependent variables are used to evaluate the medical context, such as the patient is male and 42 years old or SDoH data. Optionally, implicit variables can be included at operations 471, based on the user selected independent and dependent variables at operation 470. For example, in a medical setting, implicit variables may include the facility in which a protocol is being applied, the use of sterilized equipment, and/or a vendor of the equipment used during the treatment of the patient.

At operation 472 it can be determined whether the existing data is sufficient for protocol evaluation, in which case data is retrieved at operation 474, and an analysis is performed at operation 490 on the existing, and optionally additional data. The operation 490 can include assignment of a predictive outcome for the protocol. The predictive outcome may be calculated and presented as a percentage, score, efficacy rating, or the like. For example, dependent variables for each protocol can be evaluated to determine the effectiveness of the protocol using a machine learning algorithm, such as c-Greedy, Greedy, PCA, or other machine learning algorithms, based on: 1) prior performance of a plurality of protocols in medical context items, 2) an expected performance of the one protocol from the plurality of protocols, 3) a counter-balanced assignment of contexts to protocols, 4) maximizing information expected to be obtained by the selection, and/or 5) other factors and techniques.

When retrieving data at operation 474 medical documents can be searched for medical context items and results, such as by using natural language processing (NLP). Such techniques may provide more information than using only formally labeled and sorted data. Within a set of medical documents, while clinicians tend to utilize a standardized approach for annotating a patient encounter, how the document is dictated, including how the sections are labeled, the order of the sections, whether section titles exist and, if so, whether the sections are explicitly marked, varies tremendously between different institutions and between doctors at the same institution. Indeed, an individual doctor's dictation patterns may vary, either based upon the type of exam or procedure they are performing, or for completely arbitrary reasons. An NLP engine may perform a regioning analysis on each document to map the variation to the standard note types and normalized region titles listed above.

Optionally, data parsed from the medical documents can be indexed to facilitate parsing for corresponding indications of medical context items. In addition, the computer system may retrieve the medical documents from memory or from a data storage system. Optionally, the medical documents can be acquired by receiving the medical documents and/or an indication of location(s) of the medical documents via a network connection.

In some embodiments, a database or library identifying ontologies of the indication of the medical context can be accessed or quantitative indications of the medical context can be identified. In other examples, the indications that correlate to the indication of the medical context received can include quantitative indications of the medical context. For example, if a medical context is defined by hypertension, quantitative indications of a medical context may include blood pressures above a defined range for a patient. In examples where the indications that correlate to the indication of the medical context include quantitative indications of the medical context, a database can be accessed to identify the quantitative indications of the medical context.

In addition, or alternative, to performing analysis on the existing data at the operation 490, a new evaluation can be performed at operation 480, such as by designing and creating techniques to collect additional data, such as SDoH data, for the operation 490. In such examples, protocols from a plurality of protocols can be selected for each medical context item. The protocols may be randomly selected or selected according to other techniques. At operation 482, an evaluation plan for the different selected protocols can be generated. The evaluation plan for the different selected protocols can be presented at operation 484. The variables selected in operation 470 can be refined based on time, repetition, or expected results indicated by the evaluation plan, at operation 486. Information related to each medical context item can be monitored, such as to collect data for the evaluation at operation 488. The collected data may be optionally combined with preexisting data, and the operation 490 can be performed on the collected data or on existing data.

After the operation 490 additional independent variables (indicators) can be connected to the evaluation at operation 491. After the operation 490 an evaluation summary for the plurality of protocols can be generated and presented at operation 492

In an example application of the techniques of FIG. 4, a medical facility may evaluate the effectiveness of different protocols for patients with varying SDoH circumstances. A user may determine if data already exists at operation 472 or may define a new evaluation protocol at operation 480. Either way, the operation 490 can be performed based on the measured variables to select which protocol would be the most effective to treat the patient based, at least in part, on the SDoH data.

Other “implicit” variables may be identified for the evaluation at operation 471, such as the hospital type or the physician's training history that could be used for further improvement and/or the identification of future studies. If the evaluation protocol already has sufficient data as determined at operation 472, that data can be extracted from a data storage system at operation 474, analyzed at operation 490, and presented to the user at operation 492. If defining a new evaluation protocol at operation 480, the conditions can be randomized and assigned to different hospitals/physicians/cleaning teams at operation 482 and an evaluation plan can be proposed at operation 484. If the user would like to edit the protocol based on time, repetition, or other needs, the user can be presented the option at operation 486 and the evaluation plan can be updated at operation 482. Data can then be collected at operation 488, and other possible indicators, from operation 491, can be connected for the analysis at operation 490. These indicators may not be directly associated with the defined measured variables, but they may help predict the outcome or play a causal role. The results can be generated and presented to the user at operation 492. This can include, but is not limited to, suggesting protocol changes based on relative probabilities of the impact of other variables. The method of communicating the protocol evaluation results can vary depending on the level of analysis or could even be tailored for each user's preference or known method of preferred follow-through (e.g., email results and reminders to user A, send daily text messages to user B, etc.).

The inclusion of SDoH data to clinical risk adjustment is an evolution of a clinical risk grouper (CRG) model, such as that described regarding FIG. 4, using statistical and analytical processes which were unavailable even 10 years ago. Categorization and organization of large datasets into classes can be done efficiently and repeatably using techniques such as latent class analysis, machine learning classifications, and other similar discrete mathematical based approaches. SDoH information from various sources can be added to an existing CRG model as, for example, a post hoc categorization of patients that further stratifies a clinical risk group into groups with varying social risks. With the continuous evolution of CRG methodologies, information from beyond just the clinical spectrum that is currently used can include SDoH information. These social determinants have a substantial literature in the public health and clinical genres supporting the influence from where a person lives and their psycho-/social network. The SDoH information can be added to the CRG data to better inform clinical risk determinants on an individual level. Inclusion of the SDoH information can increase the accuracy of the CRG system (e.g., as assessed through classification/misclassification methods, agreement statistics, etc.). SDoH data that can be included in an individual CRG assessment include select Z Codes (Factors influencing health status and contact with health services) from ICD-10 insurance claims within the range Z55-Z65, responses from users of healthcare software, such as the AssessMyHealth survey used by a 3M™ health information system (HIS) Medicaid client, among others, currently unidentified sources of individual-level SDoH data such as social support, food security, economic stability, healthcare access, language barriers, transportation, neighborhood environment, or the like,

Data directly from individuals can be beneficial for including SDoH in clinical risk assessment, this includes client claims data, specifically CRG-related elements, utilization, costs, and Potentially Preventable Event (PPE) measurements. All data can be de-identified, such that no private or confidential information is at risk. At least some embodiments can operate without protected health information (PHI). For example, data can be separated into those with Z-codes (55-65) and those without. The without group can be randomly sampled, stratified on an advanced code review group (ACRG) or some level thereof, to create a comparison group. A machine learning framework can be utilized for this analysis. A training set of the Z-code data can be used to test the modelling. The remaining Z-code data can be set as a verify data set. The sampled non-Z-code data that is subset can be used in both test and verify steps.

Given that categorical measures are being used, latent class analysis can be used to construct patient driven categorizations of Z-codes or other SDoH categories that may be defined. Latent class analysis is a statistical model-based approach that utilizes the item-response probabilities across the data and looks for commonalities based on the patterns of responses. These thematic interpretations may be defined as latent classes. Each class represents a latent variable which serves as an unobserved causal influence on the responses. These classes or clusters, can then be used as an add-on stratification to ACRG/CRG/etc., towards the goal of increasing the precision of the categorical clinical risk model. If various data types are made available then other statistical methodologies may be needed, these included but are not limited to machine learning technique and multivariable regression models.

There are many ways to include SDoH in a clinical risk analysis. Examples include, Patent Cooperation Treaty (PCT) application WO2017079047 (US2016/059315), titled “Identification of Low-Efficacy Population”, and filed on May 11, 2017, PCT application WO2017112851 (US2016/068253), titled “Health management system with multidimensional performance representation”, and filed on Jun. 29, 2017, and U.S. Pat. No. 8,571,892, titled “Method of Grouping and Analyzing Clinical Risks”, and filed on Aug. 21, 2006, the contents of which are incorporated by reference herein in their entireties. The PCT application WO2017079047 describes systems and methods for identification of low-efficacy treatments and the corresponding populations that are subject to the low-efficacy treatments. Inclusion of SDoH data can improve the identification of such low-efficacy treatments and populations. The PCT application WO2017112851 describes techniques for identification of patient diagnosis and a corresponding treatment. Inclusion of SDoH data can improve the identification of the diagnosis and treatment. The U.S. Pat. No. 8,571,892 describes systems and methods for grouping and analyzing clinical risks. Inclusion of SDoH data can improve the accuracy of such grouping and analysis.

Embodiments can provide a quantitative model-based SDoH score that can be used to assess and capture the public health environment on a broader spectrum and with greater sensitivity to factors that are associated to health inequalities. Using a technique like PCA more accurately models the real value of the SDOH data at a more granular level than previously available, such as at a census tract level. The SDoH score provides several improvements over prior SES scores, which used only SES measures such as income and education. Overall, the proposed PCA SDoH score shows greater granularity, more precise accountability of variation, more accurate scoring, and a broader range of measures than its predecessors. In one or more embodiments, a structural equation model can be used to incorporate binary variables into the PCA scoring model. The geographically defined SDoH score can be determined for a census tract and a user can use a residence address to determine the SDoH of the census tract in which a person resides. Indications of SDoH factors at an individual level can be reflected in a range of Z codes, which are now available with the advent of International Classification of Diseases (ICD-10) as supplemental diagnosis codes or information on healthcare claims, or from individual responses to surveys such as AssessMyHealth or from public or private insurers or other entities who may collect patient reported outcomes (PRO) data.

As previously discussed, the SDoH score can be calculated for census tracts within a given geographical region and provides a metric by which the user can compare census tracts to one another on the dimension of SDoH. The SDoH score can also be used as a covariate in various population health analyses. For example, a researcher can investigate any number of health services measures, such as primary care utilization, hospital readmissions, or pharmaceutical adherence, and examine SDoH as a potential explanatory or confounding variable. Furthermore, SDoH by geographic area can be overlaid on a map along with study variables or point locations of hospitals or other health care facilities to provide visual representation.

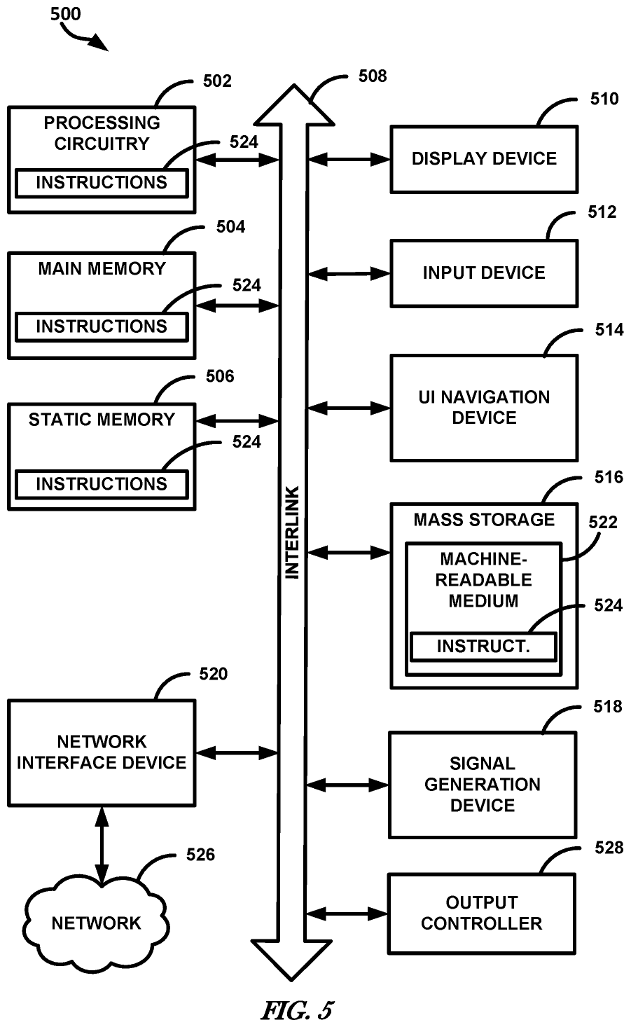

FIG. 5 illustrates, by way of example, a block diagram of an example of a device 500 upon which any of one or more processes (e.g., methods) discussed herein can be performed. The device 500 (e.g., a machine) can operate to perform at least a portion of all the method 100 or 400 discussed herein. In some embodiments, the processing circuitry 203, data ingest circuitry 204, data standardization circuitry 206, PCA circuitry 208, or the scoring circuitry 212 can include one or more of the components of the device 500. In some examples, the device 500 can operate as a standalone device or can be connected (e.g., networked) to one or more items, such as the database 202A-202C or 210. The processing circuitry 203 can include one or more of the items of the device 500, or the device 500 can implement at least a part of a middleware, cloud, distributed, or other solution to performing one or more of the methods discussed herein.

Embodiments, as described herein, can include, or can operate on, logic or a few components, modules, or mechanisms. Modules are tangible entities (e.g., hardware) capable of performing specified operations when operating. A module includes hardware. In an example, the hardware can be specifically configured to carry out a specific operation (e.g., hardwired). In an example, the hardware can include configurable execution units (e.g., transistors, logic gates (e.g., combinational and/or state logic), or other circuitry, etc.) and a computer-readable medium containing instructions, where the instructions configure the execution units to carry out a specific operation when in operation. The configuring can occur under the direction of the executions units or a loading mechanism. Accordingly, the execution units can be communicatively coupled to the computer readable medium when the device is operating. In this example, the execution units can be a user of more than one module. For example, under operation, the execution units can be configured by a first set of instructions to implement a first module at one point in time and reconfigured by a second set of instructions to implement a second module.

Device (e.g., computer system) 500 can include a hardware processor 502 (e.g., a central processing unit (CPU), a graphics processing unit (GPU), a hardware processor core, processing circuitry (e.g., logic gates, multiplexer, state machine, a gate array, such as a programmable gate array, arithmetic logic unit (ALU), or the like), or any combination thereof), a main memory 504 and a static memory 506, some or all of which can communicate with each other via an interlink (e.g., bus) 508. The device 500 can further include a display unit 510, an input device 512 (e.g., an alphanumeric keyboard), and a user interface (UI) navigation device 514 (e.g., a mouse). In an example, the display unit 510, input device 512 and UI navigation device 514 can be a touch screen display. The device 500 can additionally include a storage device (e.g., drive unit) 516, a signal generation device 518 (e.g., a speaker), and a network interface device 520. The device 500 can include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 516 can include a machine-readable medium 522 on which is stored one or more sets of data structures or instructions 524 (e.g., software) embodying or utilized by any one or more of the techniques or functions described herein. The instructions 524 can also reside, completely or at least partially, within the main memory 504, within static memory 506, or within the hardware processor 502 during execution thereof by the device 500. In an example, one or any combination of the hardware processor 502, the main memory 504, the static memory 506, or the storage device 516 can constitute machine-readable media.

While the machine readable medium 522 is illustrated as a single medium, the term “machine readable medium” can include a single medium or multiple media (e.g., a centralized or distributed database, and/or associated caches and servers) configured to store the one or more instructions 524. The term “machine readable medium” can include any tangible medium that is capable of storing, encoding, or carrying instructions for execution by the device 500 and that cause the device 500 to perform any one or more of the techniques (e.g., processes) of the present disclosure, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks. A machine-readable medium does not include signals per se.

The instructions 524 can further be transmitted or received over a communications network 526 using a transmission medium via the network interface device 520 utilizing any one of several transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 520 can include one or more physical jacks (e.g., Ethernet, coaxial, or phone jacks) or one or more antennas to connect to the communications network 526. In an example, the network interface device 520 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the device 500, and includes digital or analog communications signals or other intangible medium to facilitate communication of such software.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, although the present disclosure has been specifically disclosed by specific embodiments and optional features, modification and variation of the concepts herein disclosed may be resorted to by those of ordinary skill in the art, and that such modifications and variations are within the scope of embodiments of the present disclosure.

### Additional Embodiments

The following exemplary embodiments are provided, the numbering of which is not to be construed as designating levels of importance:

Example 1 includes a computing device to implement a model for determining a social determinant of health (SDoH) score, the computing device comprising computer program code embodied on a memory, the computer program code, when executed by processing circuitry, causes the processing circuitry to perform operations comprising receiving first data of three or more data types, each data type corresponding to an SDoH domain including economic stability, education, social and community context, health and health care, and neighborhood and built environment, the first data related to a specified geographic region, performing a principal component analysis (PCA) on the received first data to determine respective contribution values for each domain, the contribution values indicating a relative amount of variation the domain contributes to the SDoH score, receiving second data of the three or more data types, the second data related to a first sub-geographical region within the specified geographic region, and determining the SDoH score for the first sub-geographical region based on the received second data and the corresponding contribution values.

In Example 2, Example 1 further includes, wherein the operations further comprise standardizing the received first data to a common scale before performing the PCA and wherein the PCA is performed on the standardized first data.

In Example 3, Example 2 further includes, wherein standardizing the received first data includes performing a z-transformation on the received first data.

In Example 4, at least one of Examples 1-3 further includes, wherein the operations further comprise standardizing the determined SDoH score to a specified scale.

In Example 5, at least one of Examples 1-4 further includes, wherein the specified geographical region is comprised of a plurality of disjoint sub-geographical regions including the first sub-geographical region, receiving the second data includes receiving data for each sub-geographical region of the plurality of sub-geographical regions, determining the SDoH score includes determining respective SDoH scores for each of the sub-geographical regions, and the operations further include, encoding the determined SDoH scores by color and causing a display to provide a view of the specified geographical region with each of the sub-geographical regions colored consistent with the encoding.

In Example 6, at least one of Examples 1-5 further includes, wherein the data type corresponding to the health and healthcare domain includes a value indicating a proportion of a population in the sub-geographical region that has health insurance.

In Example 7, at least one of Examples 1-6 further includes, wherein the data type corresponding to the neighborhood and built environment includes data indicating one or more of how accessible healthy food is within the sub-geographical region, a quality of housing available within the sub-geographical region, air quality within the sub-geographical region, water quality within the sub-geographical region, or a relative amount of distressed or underserved geographies within the sub-geographical region.

In Example 8, at least one of Examples 1-7 further includes, wherein the data type corresponding to the social and community context includes an indication of the number of people living in the sub-geographical region.

In Example 9, at least one of Examples 1-8 further includes, wherein the operations further include, identifying the SDoH score or corresponding data corresponding to an individual user and identifying a diagnosis, treatment, or risk of re-admission based, at least in part, the SDoH score or corresponding SDoH data.

Example 10 includes a computer-implemented method for determining a social determinant of health (SDoH) score, the method including operations comprising receiving first data of three or more data types, each data type corresponding to an SDoH domain including economic stability, education, social and community context, health and health care, and neighborhood and built environment, the first data related to a specified geographic region, performing a principal component analysis (PCA) on the received first data to determine respective contribution values for each domain, the contribution values indicating a relative amount of variation the domain contributes to the SDoH score, receiving second data of the three or more data types, the second data related to a first sub-geographical region within the specified geographic region, and determining the SDoH score for the first sub-geographical region based on the received second data and the corresponding contribution values.

In Example 11, Example 10 further includes, wherein the operations further comprise standardizing the received first data to a common scale before performing the PCA and wherein the PCA is performed on the standardized first data.

In Example 12, Example 11 further includes, wherein standardizing the received first data includes performing a z-transformation on the received first data.

In Example 13, at least one of Examples 10-12 further includes, wherein the operations further comprise standardizing the determined SDoH score to a specified scale.

In Example 14, at least one of Example 10-13 further includes, wherein the specified geographical region is comprised of a plurality of disjoint sub-geographical regions including the first sub-geographical region, receiving the second data includes receiving data for each sub-geographical region of the plurality of sub-geographical regions, determining the SDoH score includes determining respective SDoH scores for each of the sub-geographical regions, and the operations further include, encoding the determined SDoH scores by color and causing a display to provide a view of the specified geographical region with each of the sub-geographical regions colored consistent with the encoding.

In Example 15, at least one of Examples 10-14 further includes, wherein the data type corresponding to the health and healthcare domain includes a value indicating a proportion of a population in the sub-geographical region that has health insurance.

In Example 16, at least one of Examples 10-15 further includes, wherein the data type corresponding to the neighborhood and built environment includes data indicating one or more of how accessible healthy food is within the sub-geographical region, a quality of housing available within the sub-geographical region, air quality within the sub-geographical region, water quality within the sub-geographical region, or a relative amount of distressed or underserved geographies within the sub-geographical region.

In Example 17, at least one of Examples 10-16 further includes, wherein the data type corresponding to the social and community context includes an indication of the number of people living in the sub-geographical region.

In Example 18, at least one of Examples 10-17 further includes, wherein the operations further include, identifying the SDoH score or corresponding data corresponding to an individual user and identifying a diagnosis, treatment, or risk of re-admission based, at least in part, the SDoH score or corresponding SDoH data.

Example 19 includes a machine-readable medium including instructions stored thereon that, when executed by a machine, cause the machine to perform the operations of one of Examples 1-18.

1. A computing device to implement a model for determining a social determinant of health (SDoH) score, the computing device comprising computer program code embodied on a memory, the computer program code, when executed by processing circuitry, causes the processing circuitry to perform operations comprising:
receiving first data of three or more data types, each data type corresponding to an SDoH domain including economic stability, education, social and community context, health and health care, and neighborhood and built environment, the first data related to a specified geographic region;
performing a principal component analysis (PCA) on the received first data to determine respective contribution values for each domain, the contribution values indicating a relative amount of variation the domain contributes to the SDoH score;
receiving second data of the three or more data types, the second data related to a first sub-geographical region within the specified geographic region; and
determining the SDoH score for the first sub-geographical region based on the received second data and the corresponding contribution values.

2. The computing device of claim 1, wherein the operations further comprise standardizing the received first data to a common scale before performing the PCA and wherein the PCA is performed on the standardized first data.

3. The computing device of claim 2, wherein standardizing the received first data includes performing a z-transformation on the received first data.

4. The computing device of claim 1, wherein the operations further comprise standardizing the determined SDoH score to a specified scale.

5. The computing device of claim 1, wherein:
the specified geographical region is comprised of a plurality of disjoint sub-geographical regions including the first sub-geographical region,
receiving the second data includes receiving data for each sub-geographical region of the plurality of sub-geographical regions,
determining the SDoH score includes determining respective SDoH scores for each of the sub-geographical regions, and
the operations further include, encoding the determined SDoH scores by color and causing a display to provide a view of the specified geographical region with each of the sub-geographical regions colored consistent with the encoding.

6. The computing device of claim 1, wherein the data type corresponding to the health and healthcare domain includes a value indicating a proportion of a population in the sub-geographical region that has health insurance.

7. The computing device of claim 1, wherein the data type corresponding to the neighborhood and built environment includes data indicating one or more of how accessible healthy food is within the sub-geographical region, a quality of housing available within the sub-geographical region, air quality within the sub-geographical region, water quality within the sub-geographical region, or a relative amount of distressed or underserved geographies within the sub-geographical region.

8. The computing device of claim 1, wherein the data type corresponding to the social and community context includes an indication of the number of people living in the sub-geographical region.

9. The computing device of claim 1, wherein the operations further include, identifying the SDoH score or corresponding data corresponding to an individual user and identifying a diagnosis, treatment, or risk of re-admission based, at least in part, the SDoH score or corresponding SDoH data.

10. A computer-implemented method for determining a social determinant of health (SDoH) score, the method including operations comprising:
receiving first data of three or more data types, each data type corresponding to an SDoH domain including economic stability, education, social and community context, health and health care, and neighborhood and built environment, the first data related to a specified geographic region;
performing a principal component analysis (PCA) on the received first data to determine respective contribution values for each domain, the contribution values indicating a relative amount of variation the domain contributes to the SDoH score;
receiving second data of the three or more data types, the second data related to a first sub-geographical region within the specified geographic region; and
determining the SDoH score for the first sub-geographical region based on the received second data and the corresponding contribution values.

11. The method of claim 10, wherein the operations further comprise standardizing the received first data to a common scale before performing the PCA and wherein the PCA is performed on the standardized first data.

12. The method of claim 11, wherein standardizing the received first data includes performing a z-transformation on the received first data.

13. The method of claim 10, wherein:
the specified geographical region is comprised of a plurality of disjoint sub-geographical regions including the first sub-geographical region,
receiving the second data includes receiving data for each sub-geographical region of the plurality of sub-geographical regions,
determining the SDoH score includes determining respective SDoH scores for each of the sub-geographical regions, and
the operations further include, encoding the determined SDoH scores by color and causing a display to provide a view of the specified geographical region with each of the sub-geographical regions colored consistent with the encoding.

14. The method of claim 10, wherein the data type corresponding to the health and healthcare domain includes a value indicating a proportion of a population in the sub-geographical region that has health insurance.

15. The method of claim 10, wherein the data type corresponding to the neighborhood and built environment includes data indicating one or more of how accessible healthy food is within the sub-geographical region, a quality of housing available within the sub-geographical region, air quality within the sub-geographical region, water quality within the sub-geographical region, or a relative amount of distressed or underserved geographies within the sub-geographical region.
